(19) United States
(12) Patent Application Publication
(10) Pub. No.: US 2008/0215071 A1
(43) Pub. Date: Sep. 4, 2008

(54) Implantable Prosthesis for Repairing Hernia Defects

(76) Inventors: Hanngorg Zimmermann, Rosstal (DE); Markus Heinlein, Gossweinstein (DE)

Correspondence Address: MCGLEW & TUTTLE, PC, P.O. BOX 9227, SCARBOROUGH STATION, SCARBOROUGH, NY, 10510-9227, US

(21) Appl. No.: 10/598,042

(22) PCT Filed: Feb. 22, 2005
(86) PCT No.: PCT/EP2005/001829

§ 371 Date: Aug. 16, 2006

(30) Foreign Application Priority Data
Feb. 26, 2004 (DE) 10 2004 009 892.1

(51) Int. Cl. A61B 17/08

(52) U.S. Cl. 606/151

(57) ABSTRACT

An implantable prosthesis for repairing hernia defects comprises a basic structure (6) which is provided with concertina pleats (3) that are parallel to a direction of extension. They are fixed by a fixing arrangement so that the basic structure (6) is approximately hourglass-shaped in a plan view.

The invention relates to an implantable prosthesis for repairing hernia defects or comparable soft tissue defects, comprising a basic structure of meshed, in particular knitted, layer material which is deformable into a plug-type insert capable of being positioned in the hernia defect.

Prostheses of the generic type, which are termed “hernia plugs” in technical jargon, come in lots of varying designs. By way of example, reference can be made to U.S. Pat. No. 5,716,408 which discloses a hernia plug that consists of conical plug elements nested into each other. By pleating, the plug elements are provided with a conical, pleated wall. In this regard, the disclosed hernia plug is particularly complicated in construction and manufacture, each element having to be pleated conically and the individual elements then having to be fitted into, and then fixed to, each other.

It is an object of the invention to embody an implantable prosthesis for hernia-defect repair in such a way that it is structured significantly less complicated without suffering any major losses of therapeutical effect and fabricable at a correspondingly low construction cost, its handleability during implantation being convenient and safe.

This object is attained by the features specified in the characterizing part of claim 1. Accordingly, the basic structure of the prosthesis is comprised of a blank that is cut from the layer material in a shape preferably ranging from round to oval and arranged in parallel concertina pleats. In this regard, as compared to the prior art, there is the advantage that only one layer of material is needed for the manufacture of the basic structure.

The concertina pleats, which are available according to the invention, are fixed only approximately centrally as related to the selected direction of extension by a fixing arrangement that passes through the pleats in such a way that the basic structure, in its undeformed position of rest, is approximately hourglass-shaped in a plan view. The waist of that configuration is produced by the fixing arrangement of the pleated layers, from where the concertina pleats extend more or less strongly toward the edge of the blank.

From a handling point of view, this configuration of a prosthesis is particularly simple, the prosthesis being seized by two fingers in the vicinity of the central fixing arrangement and deformed in the way of a plug by the remaining areas being bent up. “Gathering” the pleats in the central area of the prosthesis generates high restoring forces that act against the above-mentioned deformation, which works in favour of efficient expansion of the prosthesis in the hernia defect.

With the concertina pleats being fixed centrally, the layer material can become wider again as the distance from the fixing arrangement increases so that the hourglass configuration of the basic structure in its undeformed position of rest is produced. Being constricted, the “gusset” in the vicinity of the fixing arrangement can be seized easily by the thumb and index finger for placement into the hernia defect so that the prosthesis according to the invention offers advantages of handling to a surgeon when it is placed.

A fixing arrangement of the concertina pleats that is particularly efficient, reliable and easily placed is accomplished when a fixing thread is used in accordance with the preferred embodiments of claims 2 to 4. Preferably the fixing seam, which is produced by the thread, runs crosswise of the direction of extension of the pleats and crosswise of the principal plane of extension of the undeformed prosthesis. The fixing thread preferably consists of the same plastic material as the layer-material thread—preferably polypropylene. Then the whole prosthesis consists of entirely uniform basic material, which implies considerable simplification of approval. Additionally, with this embodiment that features a fixing seam, producing the prostheses according to the invention only requires manufacturing jobs of conventional textile fabrication, namely cutting to size, gathering and sewing. More complex manufacturing jobs, such as pleating, injection-molding or casting, preforming plane blanks into a conical basic structure and the like, are avoided. The prosthesis is soft and can be draped and also easily sewn on to hernia mesh.

In an alternative embodiment, the substantially two-dimensional basic structure of the prosthesis can be shaped into a three-dimensional, plug-type configuration by the neighbouring lateral-edge areas on both sides of the constriction being connected preferably by a seam of the same thread material as the layer-material thread. In this regard, the “purity of grades” of the materials used in the prosthesis is maintained.

In keeping with another preferred embodiment, the implantable prosthesis is equipped with a continuous biocompatible coating in the form of surface metallization, preferably a coating containing titanium. As a result, the hernia plug according to the invention is particularly well tolerated. Another advantage of surface metallization resides in the accompanying hydrophilizing effect on the mesh of the plug, as a result of which the plug, as it were, gets sucked to the walls of the hernia defect it is meant to support. Mechanical expansion still improves the fixed arrangement of the hernia plug in the defect.

Finally, fabrication of the hernia plug from single-layer mesh material in combination with surface metallization has the advantage that the entire mesh surface is kept comparatively well accessible in spite of being pleated so that the metallization process, for instance by the aid of a plasma-enhanced chemical vapour deposition process as known from DE 199 45 299 A, can be implemented, covering the entire surface and resulting in a complete metallization layer on the plastic material mesh. This still improves the tissue compatibility of the hernia plug.

Finally, in another preferred embodiment, provision is made for the layer material of the basic structure to be cut to size by the aid of a cutting laser. With this being based on a thermal melting process, there is the advantage that a cleanly melted marginal area is obtained instead of “fringes” detaching at the edges.

For a stronger hernia plug of greater inherent stability to be produced, it can be provided, in keeping with another preferred embodiment, that two basic structures, which are arranged and fixed in concertina pleats, are placed crosswise one on top of the other and connected to each other so that the basic configuration of the prosthesis is substantially trefoiled.

Furthermore, when the central pleats are gathered and stitched, excessive bulking in the central area of the prosthesis can advantageously be prevented by use of a blank of oval basic shape provided with lateral constrictions.

Further features, details and advantages of the subject matter of the invention will become apparent from the ensuing description of exemplary embodiments, taken in conjunction with the attached illustrations, in which

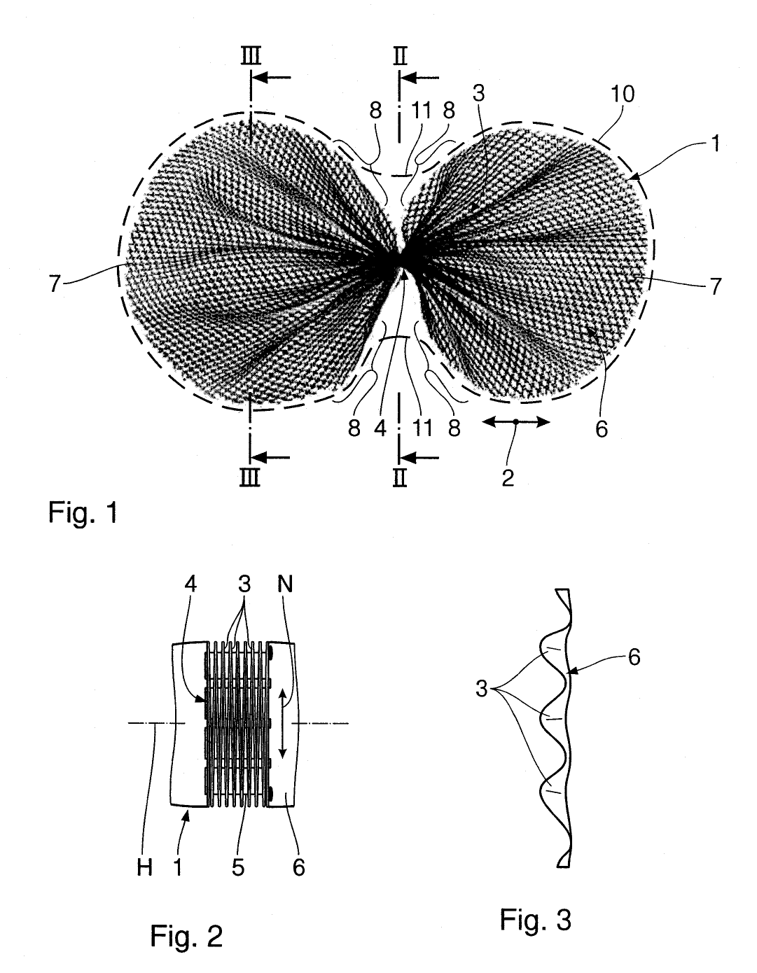

FIG. 1 is a plan view of a hernia plug;

FIG. 2 is a diagrammatic cross-sectional view on the line II-II of FIG. 1;

FIG. 3 is a diagrammatic cross-sectional view on the line III-III of FIG. 1;

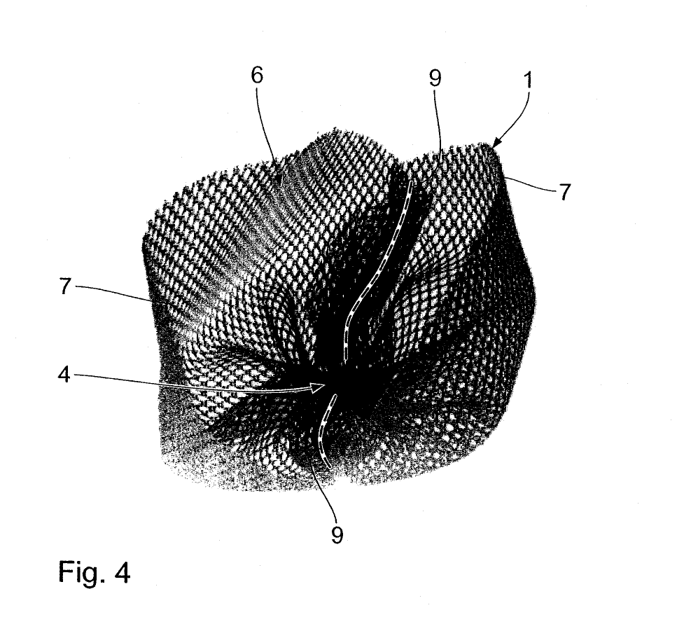

FIG. 4 is a perspective view of a second embodiment of a hernia plug.

As seen in FIG. 1, the hernia plug illustrated is comprised of meshed layer material 1 which is worked into warp-satin texture by warp-knitting from a polypropylene monofilament of a thickness of 100 dtex. The grammage of that layer material 1 is approximately 60 to 65 g/m². A centrally hourglass-shaped blank 10 of oval basic shape with lateral constrictions 11—shown by a dashed line in FIG. 1—is produced there-from by laser-beam cutting from corresponding sheet material. That blank is arranged in concertina pleats 3 in a direction of longitudinal extension 2 that is parallel to the longitudinal axis of the oval basic shape, as diagrammatically illustrated in FIG. 2. As a result of the constrictions 11 in the blank 10, the pleats 3, when gathered, do not bulk as strongly as if shaped completely ovally. For the concertina pleats 3 to be fixed, a fixing seam 4 is then stitched by the aid of a monofilament thread 5 centrally as related to the direction of longitudinal extension 2, passing through the pleated layers. The thread 5 is identical with the filament material that has been worked into the layer material 1 i.e., it consists of the same polypropylene granules and has an identical thread thickness of 100 dtex. The direction of sewing N runs crosswise of the longitudinal extension 2 (FIG. 1) and of the principal plane H of the basic structure 6 (FIG. 2).

In a plan view, the basic structure 6, fabricated as described above, is approximately hourglass-shaped in its undeformed position of rest—as seen in FIG. 1. Toward the longitudinal ends 7 of the basic structure 6, the centrally fixed concertina pleats 3 softly extend in more or less two-dimensional configuration (see FIG. 3).

After fabrication, the basic structure 6 is provided with a titanium coating by means of a prior art plasma enhanced chemical vapor deposition process; the coating covers the entire surface, its specification being detailed in the introductory part of the description.

Upon clinical use for hernia-defect repair, the basic structure 6 is seized in the vicinity of the gusset formed by the fixing seam 4 and pushed into the hernia opening. The basic structure deforms into a plug-type insert and expands in the opening owing to the restoring forces that are in particular inherent to the pleats.

The embodiment, illustrated in FIG. 4, of the hernia plug is directly produced from the embodiment according to FIGS. 1 to 3 by the adjoining lateral-edge areas, designated by the reference numeral 8 in FIG. 1, being overlapped by deformation of the basic structure 6 and one stitched to the other in a seam 9 (roughly outlined by dashes in FIG. 4). Thus the basic structure is fixed in the plug-type configuration in which it is used for hernia-defect repair. The thread material of the seam 9 is identical with the material used for the fixing seam 4 in the vicinity of the concertina folds 3, as a result of which this embodiment also consists of uniform material.

After being shaped, the configuration, illustrated in FIG. 4, of the hernia plug is provided, in the way described, with a titanium-containing coating of a thickness of<2 μm, preferably 5 to 700 nm. Practical values of coating thickness are in the range of 20 to 30 nm.

1. An implantable prosthesis for repairing hernia defects, comprising a basic structure of meshed, in particular knitted, layer material which is deformable into a plug-type insert capable of being positioned in the hernia defect,
wherein the basic structure is a blank of the layer material of a shape preferable ranging from round to oval, which is placed in concertina pleats that are parallel to a direction of extension to form pleated layers; and
wherein the basic structure, in its undeformed position of rest, is approximately hourglass-shaped in a plan view.

2. A prosthesis according to claim 1, wherein the fixing arrangement is a fixing thread that is stitched through the pleated layers.

3. A prosthesis according to claim 2, wherein a fixing seam that is formed by the fixing thread runs crosswise of a direction of extension of the pleats and crosswise of a principal plane of extension of the undeformed prosthesis.

4. A prosthesis according to claim 2, wherein the thread of layer material and the fixing thread consist of a same plastic material, preferably polypropylene, and have a same thread thickness, preferably 100 dtex.

5. A prosthesis according to claim 1, wherein the basic structure is fixed in its deformed, plug-type configuration by connection of adjoining lateral-edge areas.

6. A prosthesis according to claim 5, wherein the connection of the adjoining lateral-edge areas is produced by a seam, preferably of the same thread material as the layer-material thread.

7. A prosthesis according to claim 1, comprising a metal-containing, continuous, biocompatible coating.

8. A prosthesis according to claim 7, wherein the coating is a titanium-containing coating of a thickness of less than 2 μm, preferably 5 to 700 nm.

9. A prosthesis according to claim 1, wherein the layer material for the basic structure is laser-beam cut to size.

10. A prosthesis according to claim 1, wherein two basic structures are placed crosswise one on top of the other and joined to each other.

11. A prosthesis according to claim 1, wherein the blank, not yet pleated, of the basic structure has an outer contour ranging from round to oval with a constriction of hourglass shape in the central area.
